(19) United States
(12) Patent Application Publication
(10) Pub. No.: US 2024/0382561 A1
(43) Pub. Date: Nov. 21, 2024

(54) GLUCOREGULATORY COMPOUND, COMPOSITION AND USES THEREOF

(71) Applicant: THE HONG KONG POLYTECHNIC UNIVERSITY, Hong Kong (CN)

(72) Inventors: Chi Ming WONG, Hong Kong (CN); Tak Ho LO, Hong Kong (CN)

(21) Appl. No.: 18/693,224

(22) PCT Filed: Dec. 1, 2022
(86) PCT No.: PCT/CN2022/135913

§ 371 Date: Mar. 19, 2024

Related U.S. Application Data
(60) Provisional application No. 63/264,858, filed on Dec. 3, 2021.

(51) Int. Cl. A61K 38/17; A61P 3/10

(52) U.S. Cl. CPC A61K 38/1709; A61P 3/10

(57) ABSTRACT

The present invention provides an isolated glucoregulatory compound having both insulinotropic and blood glucose lowering activities, a composition and uses thereof for treating diseases associated with insulin deficiency or failure in glucose uptake independent of insulin secretion by pancreatic cells such as hyperglycemia, type 1 diabetes, or type 2 diabetes mellitus in a subject.

### CROSS-REFERENCE TO RELATED APPLICATION

This application claims priority from the U.S. provisional patent application Ser. No. 63/264,858 filed Dec. 3, 2021, and the disclosure of which is incorporated herein by reference in its entirety.

### REFERENCE TO SEQUENCE DISCLOSURE

A Sequence listing file with a file name “P23627PCT00_Sequence_listing.xml” in ST.26 XML file format having a file size of 8 KB created on Dec. 1, 2022 is incorporated herein by reference in its entirety.

### TECHNICAL FIELD

The present invention relates to an isolated or substantially pure glucoregulatory compound, in particular, a glucoregulatory peptide derived from cysteine rich transmembrane module containing 1 (CYSTM1), or its derivatives, and also a composition and uses thereof.

### BACKGROUND

Cysteine rich transmembrane module containing 1 (CYSTM1), or C5orf32, is a high molecular weight peptide. It is a secretory protein in mammalian epithelial cells (FIG. 1). The CYSTM family includes human CYSTM1 and mouse CYSTM1 having 97 and 104 amino acid long peptides, respectively. They share 89.4% identity and 90.4% similarity (FIG. 2). However, studies about functional roles and potential applications of CYSTM1 are very few. Venancio and Aravind (2010) suggested that CYSTM proteins might be crucial in stress tolerance across eukaryotes. Mastrokolias et al. (2015) suggested the CYSTM1 mRNA from peripheral blood as biomarker for Huntington's disease and monitor disease progression. Geng et al. (2020) reported that the transcript of C5orf32 is up-regulated in the non-responders of intravenous immunoglobulin of Kawasaki disease patients. Ou et al. (2021) identified CYSTM1-NRG2α fusion in carcinoma of unknown origin. Du and Wang (2021) proposed that CYSTM1 can be an effective biomarker for prognosis of patients with early-stage hepatocellular carcinoma (HCC). The full potential of CYSTM1 in therapeutic use is yet to be explored.

### SUMMARY OF INVENTION

Accordingly, a first aspect of the present disclosure provides an isolated or substantially pure glucoregulatory compound having both insulinotropic and blood glucose lowering activities for treating a disease in a subject.

In certain embodiments, the isolated or substantially pure glucoregulatory compound includes protein, peptide, protein fragment, or any combination thereof.

In certain embodiments, the isolated or substantially pure glucoregulatory compound is a glucoregulatory peptide derived from cysteine rich transmembrane module containing 1 (CYSTM1).

In certain embodiments, the protein, peptide or protein fragment has at least 95%, at least 96%, at least 97%, at least 98%, or at least 99% homology to an amino acid sequence of CYSTM1.

In a second aspect, a pharmaceutical composition comprising the isolated or substantially pure glucoregulatory compound is provided for treating a disease in a subject.

In certain embodiments, the disease that the pharmaceutical composition is intended to treat include type 1 diabetes, type 2 diabetes mellitus, or any failure in glucose uptake independent of insulin secretion by pancreatic cells in the subject.

In certain embodiments, the pharmaceutical composition further comprises a pharmaceutically acceptable carrier, ester, or salt.

In certain embodiments, the isolated or substantially pure glucoregulatory compound is represented by the following formula:

HN—X—CO—R′,
or a pharmaceutically acceptable salt or zwitterion thereof, wherein R′ is selected from H, OR, and —NRR;
R for each instance is independently selected from H, a lower branched alkyl group, and an unbranched alkyl group;
X is a glucoregulatory peptide selected from an amino acid sequence of SEQ ID NO: 1 and SEQ ID NO: 2;
HN denotes an amine group at an amino terminus of the glucoregulatory peptide; and CO denotes a carbonyl group at a carboxyl terminus of the glucoregulatory peptide.

In certain embodiments, the lower branched alkyl group or unbranched alkyl group includes any branched or unbranched alkyl group with fewer than six carbon atoms.

In certain embodiments, the glucoregulatory peptide of the present invention is further protected by one or more moieties such that it possesses higher insulinotropic and blood glucose lowering activities than those of its unprotected form.

A third aspect of the present invention relates to a use of the isolated or substantially pure glucoregulatory compound or its derivatives described in the first or second aspect and various embodiments of the present invention in manufacturing a pharmaceutical composition for treating a disease in a subject in need thereof.

In certain embodiments, the diseases include type 1 diabetes, type 2 diabetes mellitus, or any failure in glucose uptake independent of insulin secretion by pancreatic cells in the subject.

In certain embodiments, the subject includes humans and other non-human mammals.

In certain embodiments, the isolated or substantially pure glucoregulatory compound is a glucoregulatory peptide derived from cysteine rich transmembrane module containing 1 (CYSTM1).

In certain embodiments, the pharmaceutical composition comprises an effective amount of the glucoregulatory peptide such that an insulinotropic response is triggered and glucose uptake is enhanced in said subject by administering the pharmaceutical composition to said subject.

In certain embodiments, the pharmaceutical composition further comprises a pharmaceutically acceptable carrier described in the first aspect and various embodiments of the present invention.

In certain embodiments, the pharmaceutical composition is administered via, or the pharmaceutical composition is formulated into a form suitable for an administration route including oral, intravenous, intramuscular, intraperitoneal, or subcutaneous administrations.

As an alternative to the use of the glucoregulatory peptide in its peptide form, a nucleic acid encoding the glucoregulatory peptide can be used to be delivered to a target cell type or tissue of the subject with or without a carrier including, but not limited to, a viral vector such as adenovirus which can express the corresponding modified glucoregulatory peptide in the target cells or tissue of the subject.

In certain embodiments, one of the derivatives of the glucoregulatory peptide is an encoding sequence together with an expression vector in the pharmaceutical composition to be administered to the subject in order to express the glucoregulatory peptide in said subject.

In certain embodiments, the encoding sequence of the glucoregulatory peptide is a nucleotide sequence selected from a deoxyribonucleic acid (DNA) sequence of SEQ ID NO: 3 and SEQ ID NO: 4, or a ribonucleic acid (RNA) sequence of SEQ ID NO: 5 and SEQ ID NO: 6.

In certain embodiments, the expression vector includes one or more of adenoviral, adeno-associated, lentiviral, and retroviral vectors.

Therefore, a further aspect of the present invention relates to the use of the nucleic acid encoding the glucoregulatory peptide and the expression vector in preparation of a therapeutic agent or composition for treating the disease associated with insulin deficiency or failure in insulin-dependent glucose uptake.

In certain embodiments, the therapeutic agent or composition comprising the nucleic acid encoding the glucoregulatory peptide with or without the expression vector that can be used as a gene therapy or vaccine in a subject suffering from insulin deficiency or failure in insulin-independent glucose uptake, or alike.

Other aspects of the present invention include a method for treating diseases in a subject comprising administering the composition comprising an effective amount of the glucoregulatory peptide or its derivatives as described herein to said subject in need thereof.

This summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. Other aspects of the present invention are disclosed as illustrated by the embodiments hereinafter.

### BRIEF DESCRIPTION OF DRAWINGS

The appended drawings, where like reference numerals refer to identical or functionally similar elements, contain figures of certain embodiments to further illustrate and clarify the above and other aspects, advantages and features of the present invention. It will be appreciated that these drawings depict embodiments of the invention and are not intended to limit its scope. The invention will be described and explained with additional specificity and detail through the use of the accompanying drawings in which:

FIG. 1 shows a western blot analysis of subcellular localization of CYSTM1 and GAPDH in total lysate, insoluble fraction, nuclear fraction, cytosolic fraction, membrane bound organelle fraction and culture medium in HEK293 cells overexpressing CYSTM1;

FIG. 2 shows an amino acid sequence alignment between mouse and human CYSTM1: boxed regions represent highly conversed amino acids among CYSTM1 family; underlined sequence represents a predicted transmembrane domain;

FIGS. 3A-3F show physiological changes after acute intraperitoneal injection (FIGS. 3A-C) and oral gavage (FIGS. 3D-F) of a mouse CYSTM1 in an obese mouse model according to certain embodiments of the present invention: FIG. 3A shows glucose tolerance test (GTT) results in terms of blood glucose concentration; FIG. 3B shows a circulating serum insulin concentration; FIG. 3C shows CYSTM1 kinetics in terms of circulating CYSTM1 concentration following dose-dependent intraperitoneal injection administration of 0.5 mg/kg body weight (BW) and 1 mg/kg BW of CYSTM1 or PBS (placebo) to obese mice (n=5) over 90 min; FIG. 3D shows GTT results in terms of blood glucose concentration; FIG. 3E shows a circulating serum insulin concentration; FIG. 3F shows CYSTM1 kinetics in terms of circulating CYSTM1 concentration following an acute oral gavage administration of 1 mg/kg BW of CYSTM1 or PBS (placebo) in obese mice (n=5); FIG. 3G shows GTT results in terms of blood glucose concentration following an acute intraperitoneal (i.p.) injection of 1 mg/kg BW of human CYSTM1 or PBS in a diabetic mouse model (db/db mice) (n=5); data are expressed as means+SEM, n=4-5. *** p<0.001, ** p<0.01, *p<0.05 vs. control group without CYSTM1 treatment;

FIGS. 4A-4G show chronic effects of an oral composition of CYSTM1 according to certain embodiments of the present invention in an in vivo obese model: FIG. 4A shows a schematic diagram depicting an experimental design of administering 1 mg/kg BW CYSTM1 versus saline (control) once daily over a 25-day time course to high fat diet-induced obese (HFD) mice (n=5 per group); FIG. 4B shows the change in body weight over 21 days during the time course; FIG. 4C shows feeding blood glucose levels over 21 days during the time course; FIG. 4D shows circulating insulin levels on day 0 and day 10; FIG. 4E shows GTT results in terms of blood glucose level following oral gavage of CYSTM1 peptide on day 20; FIG. 4F shows a percentage change in blood glucose level as shown in FIG. 4E; mean±SEM, *p<0.001, ** p<0.01, *p<0.05 vs. control group without CYSTM1 treatment;

FIGS. 5A-5H show chronic effects of an adenovirus-mediated CYSTM1 (Adeno-CYSTM1) expression vector administered to an in vivo obese model according to certain embodiments of the present invention: FIG. 5A shows a schematic diagram depicting an experimental design of administering Adeno-CYSTM1 versus an adenovirus-mediated GFP vector (Adeno-GFP) as a control group on day 0 in HFD mice (n=5-6 per group) over a 14-day time course; FIG. 5B shows the body weight of the mice recorded once every 3 days during the time course; FIG. 5C shows feeding blood glucose levels in the mice recorded once every 3 days during the time course; FIG. 5D shows the circulating CYSTM1 levels on day 0 and day 10; FIG. 5E shows circulating insulin levels on day 0 and day 10; FIG. 5F shows GTT results in terms of blood glucose level on day 7; FIG. 5G shows a percentage change in blood glucose level as shown in FIG. 5F; FIG. 5H shows insulin tolerance test (ITT) results in terms of blood glucose level obtained on day 10; FIG. 5I shows a percentage change in blood glucose level as shown in FIG. 5H; mean±SEM, *** p<0.001, ** p<0.01, *p<0.05 vs. control group without Adeno-CYSTM1 transfection;

FIGS. 6A-6C show the change in insulin secretion from different pancreatic cells in a low (5.6 mM) and high (16.7 mM) glucose level treated by different concentrations of CYSTM1: FIG. 6A: MIN6 cells; FIG. 6B: β-TC-6 cells; FIG. 6C: mouse-isolated pancreatic islets (n=5-6 per cell type);

FIGS. 7A-7M show different effects on an in vivo diabetes model between inducing adenovirus-mediated CYSTM1 expression in the diabetes model (FIGS. 7A-7I) and administration of oral form of CYSTM1 peptide composition to the same diabetes model (FIGS. 7J-7M): FIG. 7A shows a schematic diagram depicting an experimental design of inducing adenovirus-mediated CYSTM1 (Adeno-CYSTM1) expression versus an expression of adenovirus-mediated GFP (Adeno-GFP) as a control group in standard chow diet (STC) male mice with streptozotocin (STZ)-induced diabetes over a 12-day time course (n=5); FIG. 7B shows the body weight of the mice over 10 days during the time course as shown in FIG. 7A; FIG. 7C shows feeding blood glucose levels in the mice over 10 days during the time course as shown in FIG. 7A; FIG. 7D shows circulating CYSTM1 levels on day 0 and day 10 during the time course as shown in FIG. 7A; FIG. 7E shows circulating insulin levels on day 0 and day 10 during the time course as shown in FIG. 7A; FIG. 7F shows GTT results in the mice on day 17; FIG. 7G percentage change of blood glucose level as shown in FIG. 7F; FIG. 7H shows ITT result in terms of blood glucose level obtained on day 20; FIG. 7I shows a percentage change in the blood glucose level as shown in FIG. 7H; FIG. 7J shows a schematic diagram depicting an experimental design of oral gavage of CYSTM1 (1 mg/kg BW) versus saline as a control in STZ-induced diabetic male mice (n=5); FIG. 7K shows the body weight of the mice over the time course as shown in FIG. 7J; FIG. 7L shows the feeding blood glucose levels of the mice as shown in FIG. 7J; FIG. 7M shows GTT result in terms of blood glucose levels obtained on day 14 from the mice as shown in FIG. 7J;

FIGS. 8A-8D show the effects of CYSTM1 on glucose uptake in 3T3-L1 adipocytes and mouse primary adipocytes: FIG. 8A shows CYSTM1 induces glucose uptake in 3T3-L1 adipocytes (n=6); FIG. 8B shows an increase in glucose uptake induced by CYSTM1 (1 μM) in 3T3-L1 adipocytes, which is inhibited by the dose-dependent manner of ERK1/2 (ERK1/2 inhibitor 1, 1 μM, 5 μM, 10 μM), AKT (GSK690693, 1 μM, 5 μM, 10 μM) and AMPK inhibitors (Dorsomorphin, 1 μM, 5 μM, 10 μM) (n=6); FIG. 8C shows that CYSTM1 induces glucose uptake in mouse primary adipocytes (n=6); FIG. 8D shows an increase in glucose uptake induced by CYSTM1 (1 μM) in mouse primary adipocytes, which is inhibited by the dose-dependent manner of ERK1/2 (ERK1/2 inhibitor 1, 1 μM, 5 μM, 10 μM), AKT (GSK690693, 1 μM, 5 μM, 10 μM) and AMPK inhibitors (Dorsomorphin, 1 μM, 5 μM, 10 μM) (n=6).

FIG. 9 shows the change in insulin secretion and glucose-induced insulin secretion of glucagon-like peptide 1 (GLP-1) by pancreatic cell MIN6 under 5.5 mM and 16.7 mM glucose conditions and treated with 10 nM GLP-1R antagonist, exendin (9-39), and/or 10 nM CYSTM1; data are expressed as mean±SEM (n=3-4); N.S.: not significant; *** p<0.001, ** p<0.01, *p<0.05 vs. control group without stimulation.

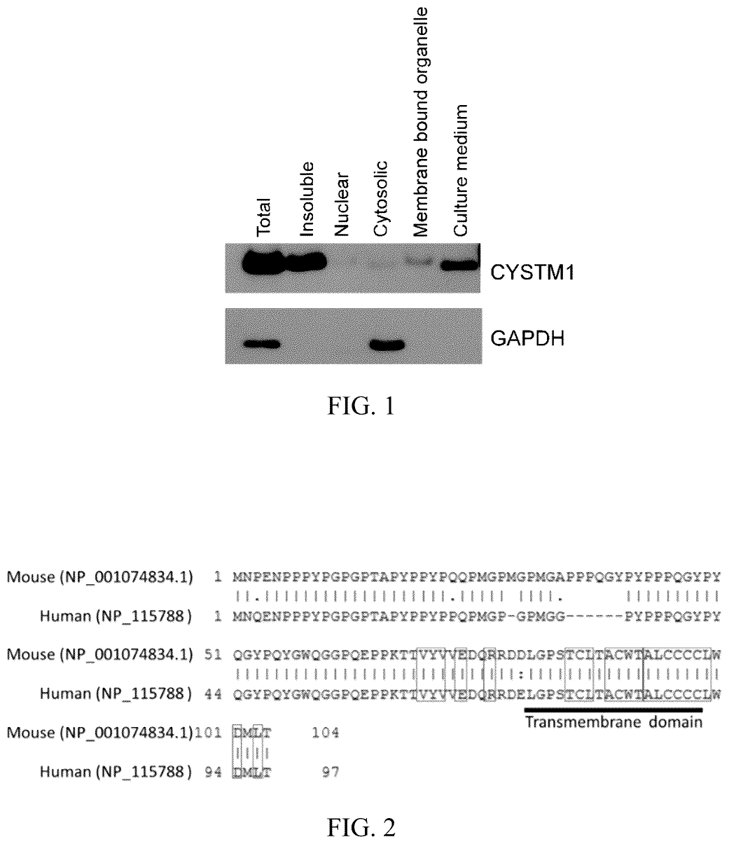

Skilled artisans will appreciate that elements in the figures are illustrated for simplicity and clarity and have not necessarily been depicted to scale.

### DETAILED DESCRIPTION OF THE INVENTION

It will be apparent to those skilled in the art that modifications, including additions and/or substitutions, may be made without departing from the scope and spirit of the invention. Specific details may be omitted so as not to obscure the invention; however, the disclosure is written to enable one skilled in the art to practice the teachings herein without undue experimentation.

The present invention provides an isolated or substantially pure glucoregulatory compound that can be a protein, peptide, protein fragment, or any combination thereof.

Preferably, the isolated or substantially pure glucoregulatory compound in various embodiments is a glucoregulatory peptide originating from CYSTM1. The CYSTM1 of the present invention can be represented by an amino acid sequence of SEQ ID NO: 1 and SEQ ID NO: 2.

Alternatively, the compound can include a nucleic acid capable of encoding the glucoregulatory peptide with or without an expression system for expressing thereof in a host cell.

In certain embodiments, the expression system is a viral vector with an insert of the corresponding encoding sequence.

Preferably, the nucleic acid capable of encoding the glucoregulatory peptide has a nucleotide sequence selected from a DNA sequence of SEQ ID NO: 3 and SEQ ID NO: 4, or an RNA sequence of SEQ ID NO: 5 and SEQ ID NO: 6.

The expression vector according to certain embodiments includes one or more of adenoviral, adeno-associated, lentiviral, and retroviral vectors. Preferably, adenoviral vector is selected as an expression system for the glucoregulatory peptide encoding sequence to express the corresponding peptide in the host cells, and the host is selected from human or non-human mammal.

In certain embodiments, in the absence of an expression vector, the nucleic acid encoding the glucoregulatory peptide can include a mRNA being directly delivered as a vaccine to a subject in need thereof to express the glucoregulatory peptide in the corresponding host cell or tissue.

The present invention also provides a pharmaceutical composition comprising the glucoregulatory peptide, or its derivatives, including any pharmaceutically acceptable carrier, thereof. In certain embodiments, the isolated or substantially pure glucoregulatory compound or its derivatives is/are represented by the following formula:

HN—X—CO—R′,
or a pharmaceutically acceptable salt or zwitterion thereof, wherein R′ is selected from H, OR, and —NRR,
R for each instance is independently selected from H, a lower branched alkyl group and an unbranched alkyl group;
X is a glucoregulatory peptide selected from an amino acid sequence of SEQ ID NO: 1 and SEQ ID NO: 2;
HN denotes an amine group at an amino terminus of the glucoregulatory peptide; and CO denotes a carbonyl group at a carboxyl terminus of the glucoregulatory peptide.

In certain embodiments, the lower branched alkyl group or unbranched alkyl group includes any branched or unbranched alkyl group with fewer than six carbon atoms.

The glucoregulatory peptide or pharmaceutical composition of the present invention is substantially free of natural contaminants and has an insulinotropic activity and blood glucose lowering activity.

In certain embodiments, the pharmaceutical composition comprises the glucoregulatory peptide or its derivatives in a concentration of 10-10 M or more in order to trigger an insulinotropic response in a subject.

In certain embodiments, the glucoregulatory peptide or its derivatives can be synthesized by any known methods including a solid-phase peptide synthesis described by Merrifield, J. M. (Chem. Soc. 85:2149 (1962)), Stewart a Young (Solid Phase Peptide Synthesis (Freeman, San Francisco, 1969), pages 27-66), or purification from cells overexpressing CYSTM1 by recombinant DNA technologies (Maniatis, T, et al., Molecular Biology: A Laboratory Manual, Cold Spring Harbor, New York (1982)), which are incorporated by reference herein. Other known methods include protein fragmentation of the naturally occurring protein sequence, if appropriate.

As used herein, the term “isolated” in connection with a compound described herein means the compound is not in a cell or organism and the compound is separated from some or all of the components that typically accompany it in a cell or organism.

As used herein, the term “substantially pure” in connection with a sample of a compound described herein means the sample contains at least 60% by weight of the compound. In certain embodiments, the sample contains at least 70% by weight of the compound; at least 75% by weight of the compound; at least 80% by weight of the compound; at least 85% by weight of the compound; at least 90% by weight of the compound; at least 95% by weight of the compound; or at least 98% by weight of the compound.

The term “derivatives” or alike of a glucoregulatory peptide described herein may refer to a compound, molecule, or moiety sharing substantial homology with their naturally occurring form, with a similarly sized fragment to that of the naturally occurring form, functionally equivalent, similar, or even superior to a glucoregulatory hormone, and/or having higher insulinotropic and blood glucose lowering activities than those of the naturally occurring form. For instance, one or more residues in the amino acid sequence of the naturally occurring CYSTM1 can be substituted with other residue(s), e.g., lysine is substituted with arginine, or isoleucine is substituted with leucine, or tyrosine is substituted with phenylalanine. These homologs may have at least 95% homology to their naturally occurring peptide, which are still expected to have similar or even substantially equivalent efficacy. In some cases, having the same insulinotropic activity exerted on target cells or tissue, one or more of these derivatives may only be in a concentration of 10-11M, which is about 10-fold lower in concentration than that of their naturally occurring form.

As used herein, the term “pharmaceutically acceptable salt” refers to those salts which are, within the scope of sound medical judgment, suitable for use in contact with the tissues of subjects without undue toxicity, irritation, allergic response and the like, and are commensurate with a reasonable benefit/risk ratio. Pharmaceutically acceptable salts are well known in the art. For example, Berge et al. describes pharmaceutically acceptable salts in detail in J. Pharmaceutical Sciences (1977) 66:1-19. Pharmaceutically acceptable salts of the compounds provided herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, besylate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In certain embodiments, organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

Pharmaceutically acceptable salts derived from appropriate bases include alkali metal, alkaline earth metal, ammonium and N+(C1-4alkyl)4 salts. Representative alkali or alkaline earth metal salts include sodium, lithium, potassium, calcium, magnesium, iron, zinc, copper, manganese, aluminum, and the like. Further pharmaceutically acceptable salts include, when appropriate, non-toxic ammonium, quaternary ammonium, and amine cations formed using counterions, such as halide, hydroxide, carboxylate, sulfate, phosphate, nitrate, lower alkyl sulfonate, and aryl sulfonate. Organic bases from which salts can be derived include, for example, primary, secondary, and tertiary amines, substituted amines including naturally occurring substituted amines, cyclic amines, basic ion exchange resins, and the like, such as isopropylamine, trimethylamine, diethylamine, triethylamine, tripropylamine, and ethanolamine. In certain embodiments, the pharmaceutically acceptable base addition salt is chosen from ammonium, potassium, sodium, calcium, and magnesium salts.

The term “pharmaceutically acceptable salts” described herein may also refer to the relatively non-toxic, inorganic and organic acid addition salts of the glucoregulatory compound of the present disclosure. These salts can be prepared in situ in the administration vehicle or the dosage form manufacturing process, or by separately reacting a purified glucoregulatory compound of the invention in its free base form with a suitable organic or inorganic acid, and isolating the salt thus formed during subsequent purification. Representative salts include the bromide, chloride, sulfate, bisulfate, carbonate, bicarbonate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like.

The pharmaceutically acceptable salts of the glucoregulatory compound of the present disclosure includes the conventional nontoxic salts or quaternary ammonium salts of the compound, e.g., from nontoxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

The glucoregulatory compound of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of the compound of the present invention. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like.

The present invention further provides a use of the glucoregulatory peptide or its derivatives in manufacturing or preparation of a pharmaceutical composition (or a medicament) for treating a disease in a subject, where the diseases include hyperglycemia, type 1 diabetes or type 2 diabetes mellitus.

The present invention additionally provides a method for treating a disease associated with insulin-deficiency or failure in glucose uptake by certain cells in a subject comprising administering the composition comprising an effective amount of glucoregulatory peptide or its derivatives to the subject in need thereof. The administration is not limited to oral, but also includes intravenous, intraperitoneal, intramuscular, and subcutaneous routes of administration.

The pharmaceutical composition of the present disclosure may be specially formulated for administration in liquid form, including those adapted for the following: parenteral administration, for example, by intravenous as, for example, a sterile solution or suspension.

In exemplary embodiments, the subject includes human and non-human mammals.

Alternatively, the glucoregulatory peptide or its derivatives may also be delivered to said subject with an expression vector including an adenovirus-mediated expression vector containing an encoding sequence of the glucoregulatory peptide or its derivatives to be expressed in host cells or target tissues of the subject.

In certain embodiments, the glucoregulatory peptides (or derivatives) of CYSTM1, and/or nucleic acids that encoding the CYSTM1, may be used as therapeutic compositions. Such therapeutic compositions may consist solely of the glucoregulatory peptides (or derivatives), or with the nucleic acid encoding the same, although, preferably, the compositions will contain the insulinotropic peptides (or derivatives thereof) in admixture with a pharmaceutically acceptable carrier vehicle.

In certain embodiments, the composition comprising the CYSTM1 may be administered intravenously, intramuscularly, subcutaneously or orally at dosages in a range from about 1 μg/kg to 10 mg/kg body weight, or at a concentration sufficient to produce serum levels of 10⁻¹⁰ M to 10⁻¹¹ M, although a lower or higher dosage may be administered. Dosage is variable subject to various factors including, but not limited to, the severity of the condition of the subject in need thereof, for example, the severity of a patient's hyperglycemia, and upon such criteria as the patient's height, weight, sex, age, and medical history. The dose may also vary depending upon whether the composition of the invention is administered in what setting, e.g., a veterinary setting to a smaller animal or in a physician setting to a human subject.

For the purpose of parenteral administration, the composition comprising the CYSTM1 or its derivatives are preferably dissolved in distilled water and the pH-value is preferably adjusted to about 6 to 8. In certain embodiments, the composition is formulated in lyophilized form. In order to facilitate the lyophilization process, lactose may be added to the solution. Preferably, the solution is then filtered, sterilized, introduced into vials, and lyophilized. In a preferred embodiment, the composition is administered orally to a subject at the time of eating or shortly thereafter. The concentration of the CYSTM1 derivatives in these compositions, and especially the concentration of CYSTM1 whether oral or parenteral, may vary from 10⁻¹² M to 10⁻⁵ M.

Pharmaceutical compositions of the present invention suitable for parenteral administration comprise the glucoregulatory compound described herein in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain sugars (such as sucrose), alcohols, non-ionic surfactants (such as Tween 20), antioxidants, buffers, bacteriostats, chelating agents, solutes which render the formulation isotonic with the blood of the intended recipient or suspending or thickening agents.

Additional pharmaceutical methods may be employed to control the duration of action. The present composition may be formulated into a controlled release preparation which can be achieved by using certain polymers to complex or adsorb the CYSTM1 or its derivatives. The controlled release system may be enabled by selecting appropriate macromolecules (for example, polyesters, polyamino acids, polyvinyl pyrrolidone, ethylenevinylacetate, methylcellulose, carboxymethylcellulose, and protamine sulfate), the concentration of macromolecules, as well as the methods of incorporation in order to exert control release effect. Another possible method to control the duration of action of the composition is to incorporate the CYSTM1 or its derivatives into certain particles made of a copolymeric material such as polyethylene vinylacetate copolymers. Alternatively, instead of incorporating the CYSTM1 or its derivatives into the copolymeric particles, it is possible to entrap which in microcapsules which are prepared, for example, by coacervation techniques, by interfacial polymerization with hydroxymethylcellulose or gelatinmicrocapsules and poly (methylmethacrylate) microcapsules, respectively, or in colloidal drug delivery systems, for example, liposomes, albumin microspheres, microemulsions, nanoparticles, and nanocapsules or in macroemulsions. Such teachings are disclosed in Remington's Pharmaceutical Sciences (1980), which is incorporated herein by reference.

Examples of suitable aqueous and non-aqueous carriers which may be employed in the pharmaceutical composition of the present invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

It is possible to enhance the biological half-life or bioavailability of the CYSTM1 or its derivatives of the present invention, thereby increasing the retention or stability of the derivatives in a recipient, by bonding which to one or more chemical “moieties’ in order to produce a compound which can be recognized and processed within a recipient to yield a CYSTM1 derivative. The “moieties’ for bonding to the present CYSTM1 may include one or more lipids, carbohydrates, amino acid residues, etc. Preferably, a preferred “moiety’ is an amino acid residue or nucleic acid. More preferably, the “moiety’ is a peptide or oligonucleotide. The amino terminal residue of CYSTM1 is a preferred site for the bonding of the “moiety.”

The composition or pharmaceutical composition of the present invention may also contain adjuvants, such as preservatives; wetting agents, emulsifying agents and dispersing agents such as sodium lauryl sulfate and magnesium stearate; as well as coloring agents, release agents, coating agents, sweetening, flavoring and perfuming agents, preservatives, solubilizing agents, buffers and antioxidants. Prevention of the action of microorganisms upon the compounds of the present disclosure may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

To determine functional attributes and efficacy of the present glucoregulatory peptide and its derivatives, various assays are employed to test the present glucoregulatory peptide and its derivatives in different in vitro and in vivo models including glucose uptake by adipocytes or insulin secretion measurement from various pancreatic cells/tissues, physiological change and glucose/insulin tolerance tests in insulin high fat diet-induced obese mouse model, streptozotocin-induced diabetic mouse model, etc.

The following examples accompanied with corresponding drawings are intended to better illustrate various embodiments of the present invention. Scope of the present invention should be defined in the appended claims.

### Example 1—Acute Intraperitoneal Injection and Oral Gavage of Recombinant CYSTM1 Stabilize Glucose Response to Glucose Loading in Mammals

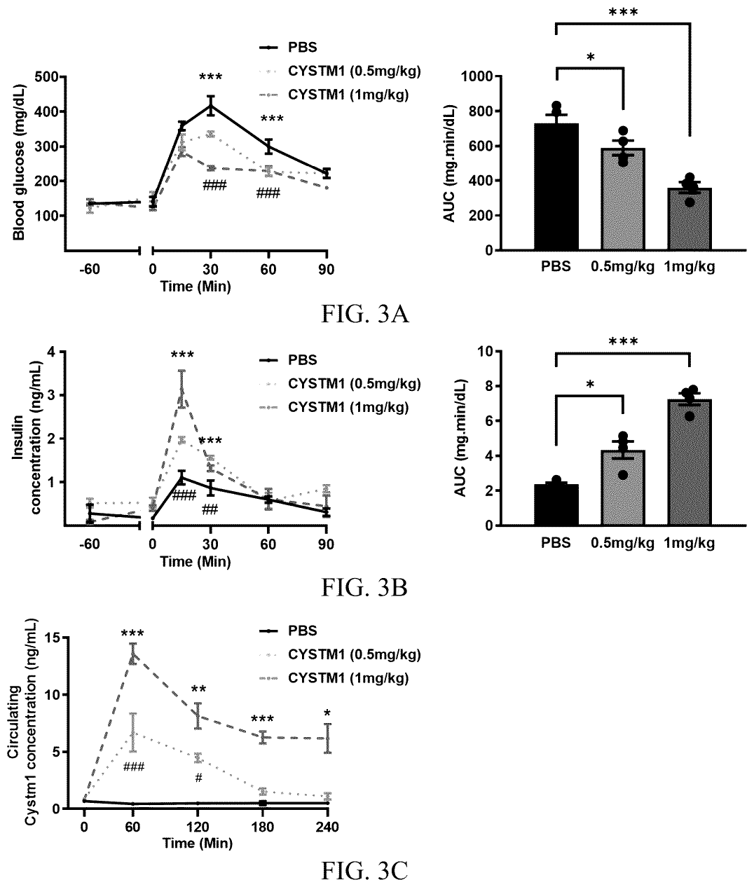

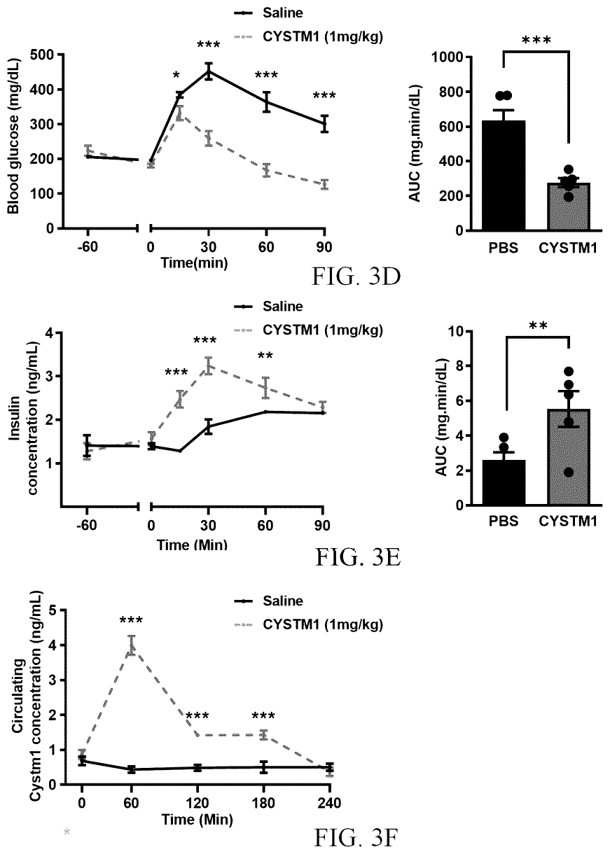

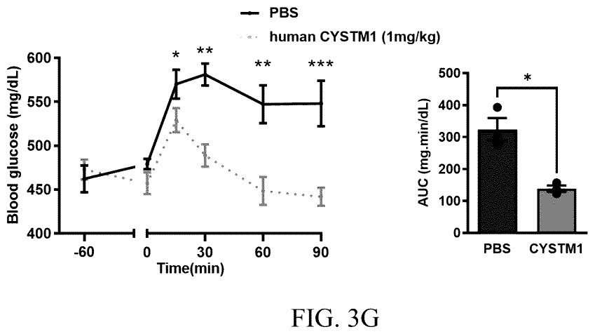

In this example, an acute intraperitoneal (i.p.) glucose tolerance test (GTT) was performed using different administration methods of delivering recombinant CYSTM1 proteins. 8-week-old male C57BL/6J mice fed a high-fat diet for 12 weeks (diet-induced obesity and diabetes mouse model) were treated with different concentrations of CYSTM1 through i.p. injection (FIG. 3A-3C) or oral administration (FIGS. 3D-3F) for 1 h following a GTT by intraperitoneal injections of 1 mg/kg glucose. Circulating CYSTM1 and insulin levels were determined by enzyme-linked immunosorbent assay (ELISA) (ImmunoDiagnostics, Hong Kong), and the glucose level was determined by a glucometer (Roche Diabetes Care, Indianapolis, IN, USA). Both administration methods can increase circulating insulin and lower glucose levels. In addition, same as mouse CYSTM1, human CYSTM1 can also lower glucose levels in db/db mice-another common mouse model of type 2 diabetes (FIG. 3G). As human and mouse CYSTM1 share high identity (89.4%) and similarity (90.4%), and human CYSTM1 appears to also lower circulating glucose in the db/db mice (a genetic mouse model of obesity and diabetes), it could be expected that human CYSTM1 may also function similarly with respect to human circulating glucose in an obese, diabetic human subject.

### Example 2—Chronically Oral Gavage of CYSTM1 Mitigates Obesity-Induced Hyperglycemia

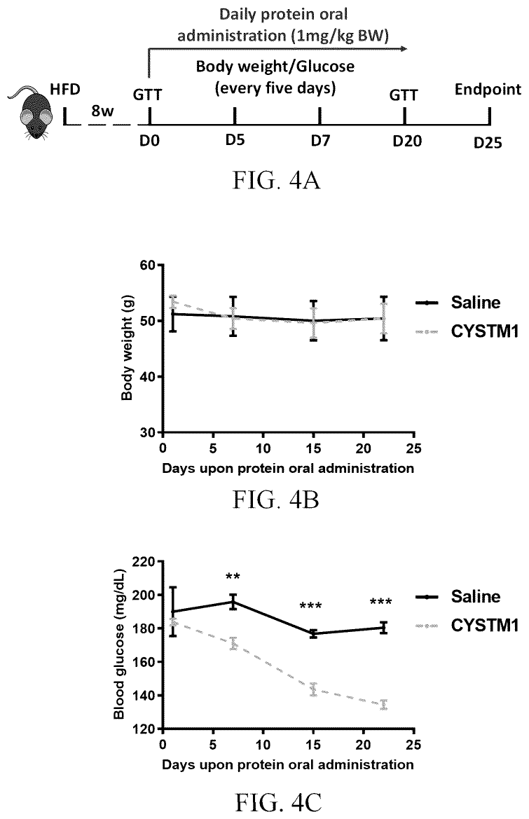

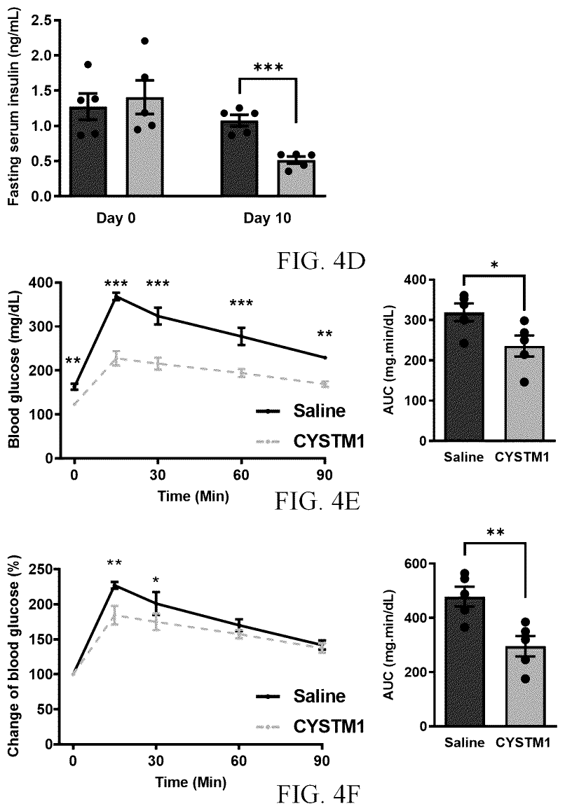

In this example, 8-week-old male C57BL/6J mice fed with a high-fat diet (HFD) were treated daily with an oral gavage of recombinant CYSTM1 protein (1 mg/kg), and the control group of mice were gavaged with saline daily. CYSTM1 treatment was carried out after 8 weeks of HFD feeding (FIG. 4A). No differences were detected in body weight between these two groups (FIG. 4B), but a significantly reduced feeding blood glucose level was observed in the CYSTM1 treatment group (FIG. 4C). A decreased level of fasting insulin concentration was shown in the CYSTM1 treatment group on day 10 when compared to the saline group (FIG. 4D). In addition, CYSTM1-treated mice exhibited better glucose homeostasis as demonstrated by IPGTT when compared to control HFD-fed mice (FIGS. 4E-4F).

### Example 3—Adenovirus-Mediated CYSTM1 (Adeno-CYSTM1) Expression Improves Obesity-Induced Hyperglycemia and Insulin Sensitivity in Mammals

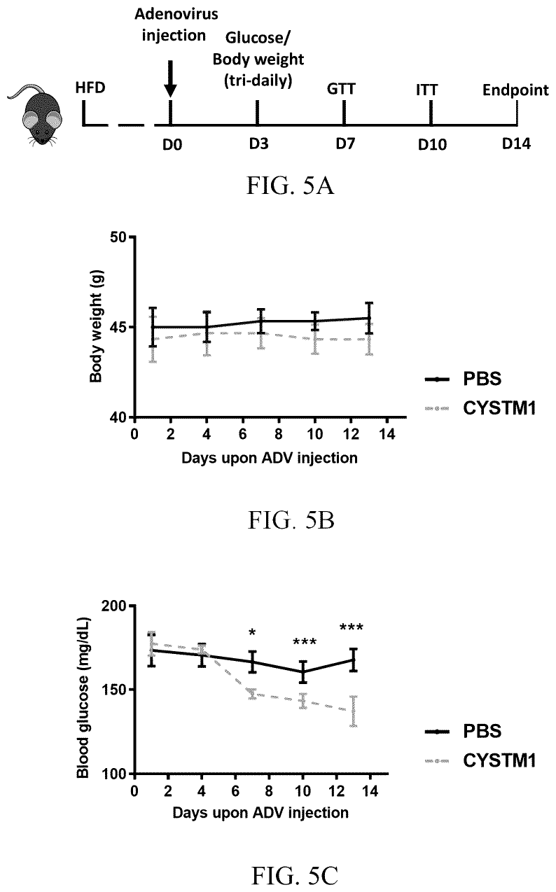

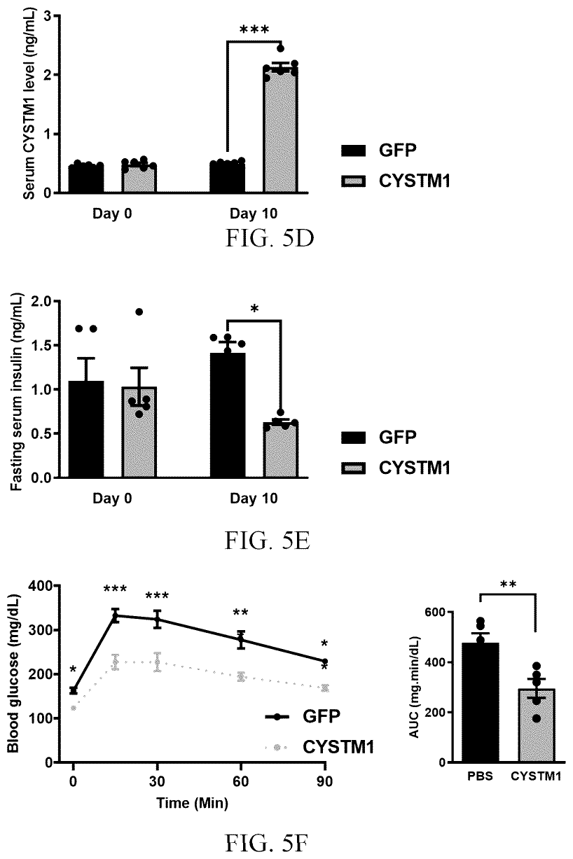

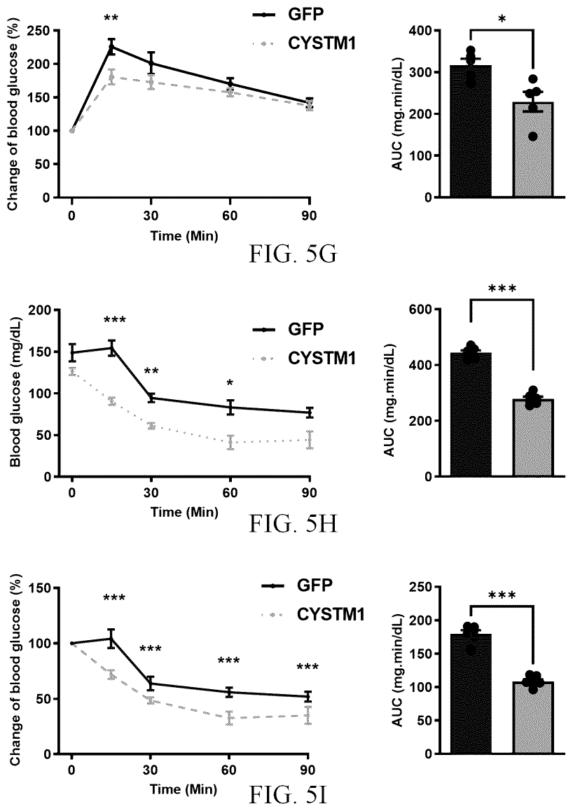

In this example, 8-week-old male C57BL/6J mice fed with HFD were infected with adenovirus expressing CYSTM1 (Adeno-CYSTM1) by a tail vein injection; mice injected with GFP were used as the control group (FIG. 5A). Treatment of Adeno-CYSTM1 did not affect the HFD-induced body weight of mice but reduced the level of feeding blood glucose (FIGS. 5B-C). Serum (circulating) CYSTM1 levels of both the CYSTM1 treatment and control groups were measured by ELISA. The serum CYSTM1 levels of adeno-CYSTM1 mice were significantly higher than those of the control group after 10 days of infection (FIG. 3D). Treatment of Adeno-CYSTM1 reduces fasting serum insulin levels as compared to the adeno-GFP group (FIG. 5E). Improved glucose homeostasis in Adeno-CYSTM1 transfected mice was demonstrated by GTT (FIGS. 5F-5G), and improvement of insulin sensitivity of Adeno-CYSTM1 transfected mice was demonstrated by insulin tolerance test (ITT) (FIGS. 5H-5I).

### Example 4—Stimulation of Insulin Secretion from Pancreatic Cells by CYSTM1

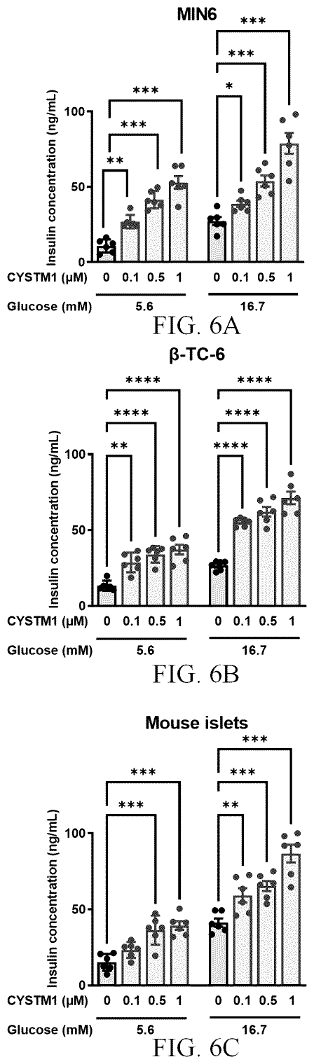

In this example, in vitro assays of insulin secretion in response to CYSTM1 treatment in low and high-glucose environments on the pancreatic cell lines (MIN6 and B-TC-6) and mouse isolated pancreatic islets were performed. Mouse insulinoma, MIN6 cells, were cultured in DMEM (Gibco) supplemented with 10% (v/v) FBS (Lonza), 100 U/mL penicillin and 100 mg/mL streptomycin, and 70 μM freshly added β-mercaptoethanol (Gibco) (37° C., 5% CO₂). B-TC-6 cells were grown in RPMI 1640 medium (Gibco) containing 5.5 mM glucose and supplemented with 10% (v/v) FBS, 100 U/mL penicillin and 100 mg/mL streptomycin, and 2 mM L-glutamine. Mouse islets were isolated from 10 to 12-week-old male C57BL/6J mice using a collagenase digestion protocol and were cultured for at least 24 h in islet medium (RPMI 1640 medium containing 11.1 mM glucose, supplemented with 10% (v/v) FBS, 100 U/mL penicillin and 100 mg/mL streptomycin before assay. On the day of assay, the medium was removed and washed with 50 μl of 1.7 mM glucose Krebs Ringer Bicarbonate Buffer (KRB: 986.82 mM NaCl, 93.88 mM KCl, 118.02 mM MgSO₄·7H2O,118.04 mM KH₂PO⁴, 833.23 mM NaHCO₃, 100 mM HEPES, and 83.93 mM CaCl₂·2H₂O containing 0.5% BSA). The KRB was immediately discarded and replaced with a fresh 100 μl of 1.7 mM glucose KRB with or without CYSTM1 in different concentrations (1 nM, 10 nM, 100 nM and 1000 nM), and the plate was incubated at 37° C. and 5% CO₂ for 45 minutes (pre-incubation step). For the fold stimulation method, the KRB was removed and replaced with another 100 μl of 5.6 mM glucose KRB (pre-incubation step 2) and incubated for another 45 min at 37° C. and 5% CO₂ before being removed and saved. For both the fold stimulation and percent secreted methods, the 5.6 mM glucose KRB was replaced with 16.7 mM glucose KRB (stimulation step) with or without CYSTM1 in different concentrations (1 nM, 10 nM, 100 nM and 1000 nM) for another 45 min. The stimulated KRB was then removed and saved. Plates were viewed under a stereomicroscope to determine whether any islets were lost during the assay; empty wells were noted and were eliminated from the final analysis. 100 μl of cell lysis buffer was added to each well. The microplates containing the decanted KRB and lysed islets were wrapped in plastic wrap and stored at −20° C. until analysis. For both cell lines, cells were resuspended in the above media and were dissociated before being seeded into 96-well plates (40,000 cells per well). Media were removed from the cells and the cells were washed twice with glucose-free KRB buffer. A measure of 100 μl of the above KRB solution containing 1.1 mM glucose was added to each well and the plates were returned to the incubator for 45 min. Subsequently, the plates were washed a further two times with glucose-free KRB and 200 μl of KRB containing glucose at low (5.6 mM) or high (16.7 mM) concentrations, with or without CYSTM1 in different concentrations (1 nM, 10 nM, 100 nM and 1000 nM). Following 45 min incubation of KRB solution, each well was removed and the insulin content of each sample was determined by a mouse insulin ELISA kit. The efficacy of CYSTM1 to stimulate insulin secretion was evaluated in each of the pancreatic cell lines: MIN6 cells (FIG. 6A), B-TC-6 cells (FIG. 6B), and isolated pancreatic islet cells (FIG. 6C). A concentration-dependent increase in insulin secretion was observed upon treatment with CYSTM1 in all assays.

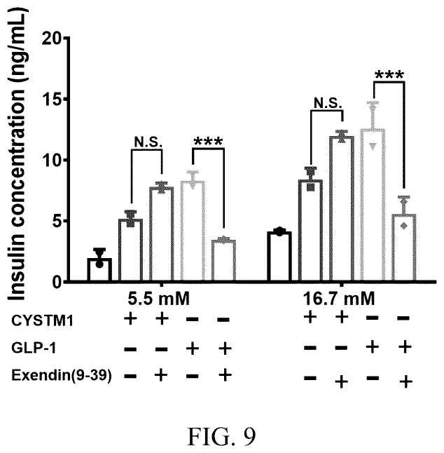

FIG. 9 also shows that CYSTM1 induces insulin secretion via GLP-1R independent pathway. Exendin (9-39), a GLP-1R antagonist, did not inhibit CYSTM1 activity in MIN6 pancreatic beta cell. Glucose-induced insulin secretion of GLP-1 (10 nM) was inhibited by exendin (9-39) (10 nM), whereas CYSTM1 (10 nM) was not affected by the presence of exendin (9-39). The results in FIG. 9 suggest that the CYSTM1 triggers the insulin secretion via GLP-1R independent pathway.

### Example 5—Adenovirus-Mediated CYSTM1 Expression and Orally Administrated CYSTM1 Improve Streptozotocin (STZ)-Induced Diabetes

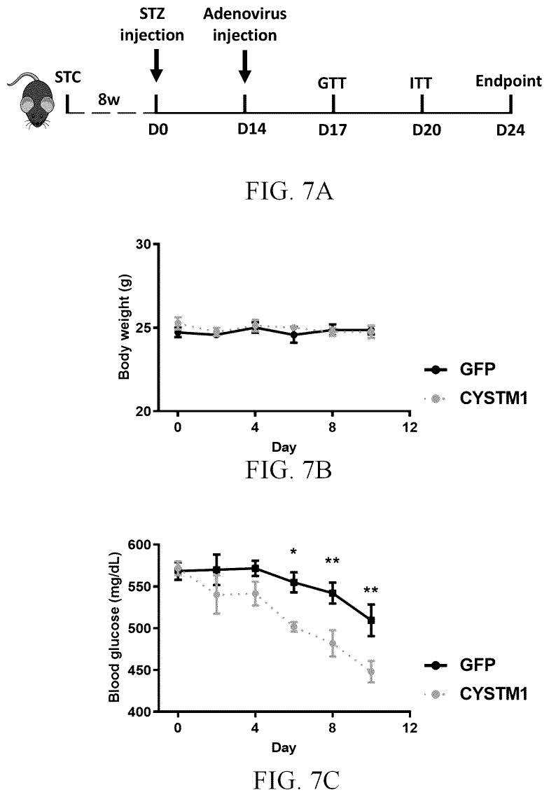

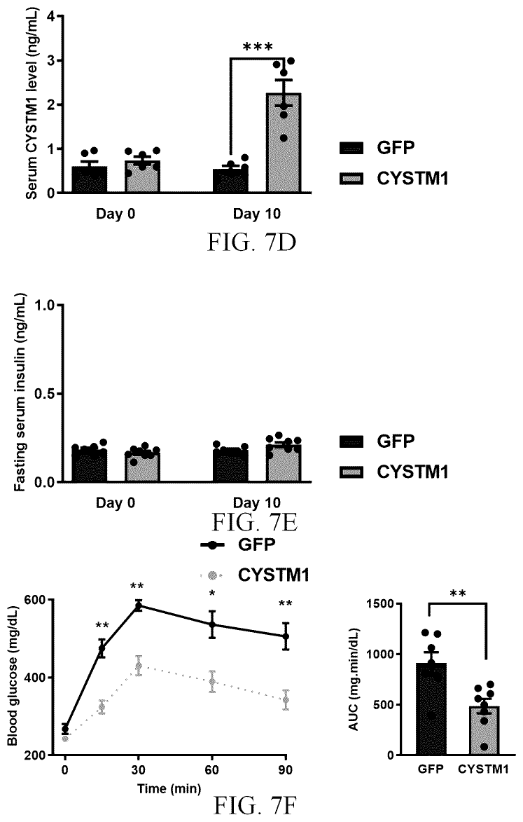

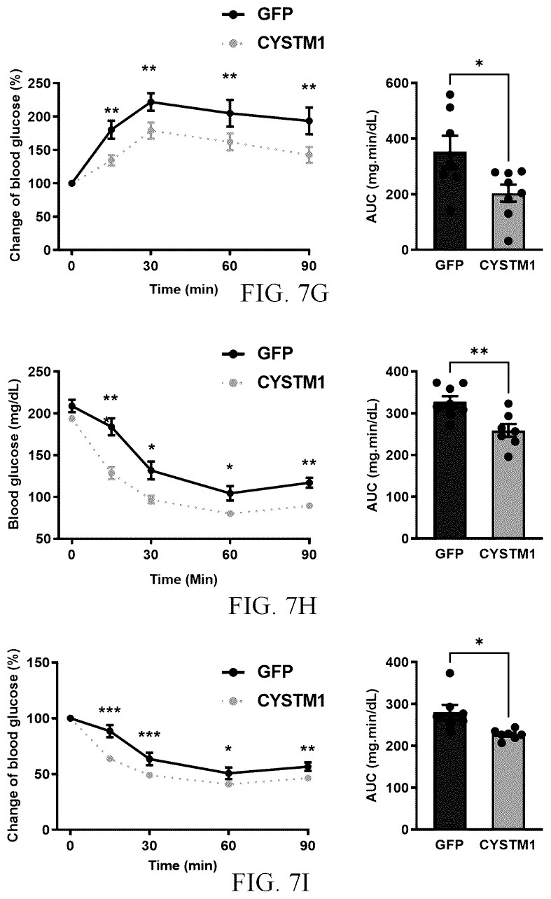

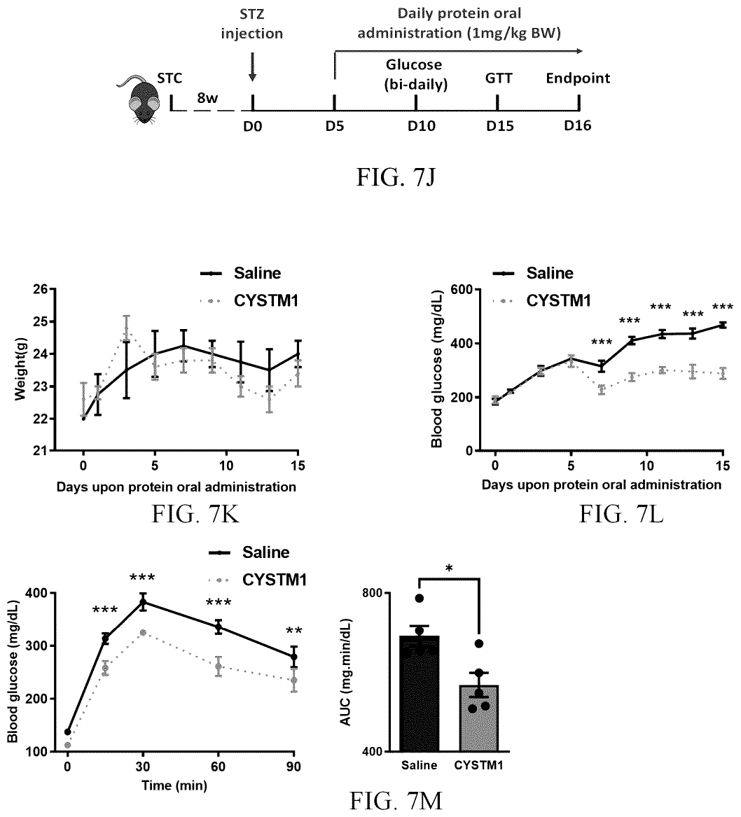

In this example, 8-week-old male C57BL/6J mice fed with standard chow (STC) diet were induced to diabetes with streptozotocin (STZ) for 14 days followed by transfection with adenovirus expressing CYSTM1 (Adeno-CYSTM1) via a tail vein injection. Mice injected with adenovirus expressing GFP (Adeno-GFP) were used as the control group (FIG. 7A). Treatment of Adeno-CYSTM1 did not affect the body weight of the STZ-induced diabetic mice but reduced the levels of feeding blood glucose (FIGS. 7B-7C). Serum (circulating) CYSTM1 levels of both the Adeno-CYSTM1-treatment and the control group were measured by ELISA. The serum CYSTM1 levels of Adeno-CYSTM1 mice were significantly higher than the control group after 10 days of transfection (FIG. 7D). The fasting insulin level was not altered in both groups after the STZ injection (FIG. 7E). Improved glucose homeostasis in Adeno-CYSTM1 transfected mice was demonstrated by GTT (FIGS. 7F-7G), and improvement of insulin sensitivity of Adeno-CYSTM1 infected mice was demonstrated by ITT (FIGS. 7H-7I).

Turning to FIG. 7J, standard chow diet (STC) mice were injected with streptozotocin (STZ) to induce diabetes for five days and then treated with either CYSTM1 protein (1 mg/kg) or saline daily. The group of mice treated with saline were regarded as the control group. A similar pattern was found in this group of mice, where no difference was detected in body weight (FIG. 7K) but a significantly lower feeding blood glucose level was found in the orally administered CYSTM1 group (FIG. 7L). Furthermore, IPGTT results on day 15 suggested that the orally administered CYSTM1 group protects from STZ-induced glucose dysregulation (FIG. 7M).

### Example 6—CYSTM1 Increases Glucose Uptake in Adipocytes

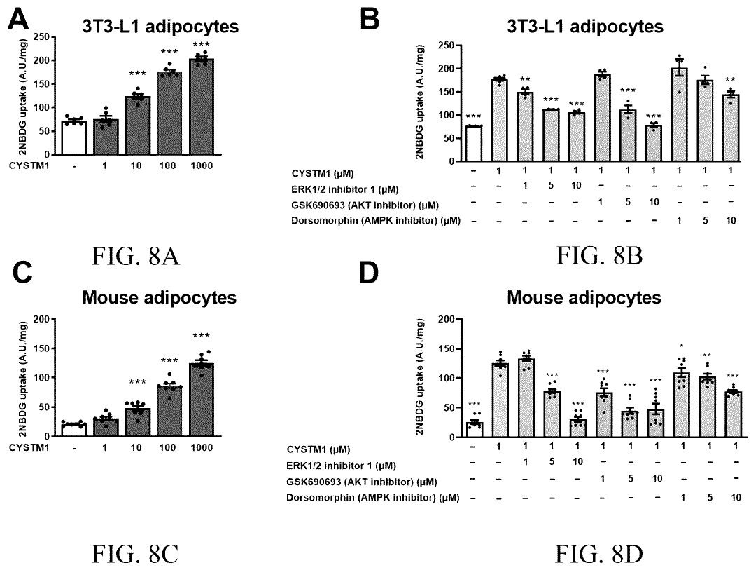

To determine whether CYSTM1 can increase glucose uptake in adipocytes, in vitro assays of 2-NBDG uptake in response to CYSTM1 treatment and potential inhibitors in 3T3-L1 adipocytes and mouse primary adipocytes were performed. 3T3-L1 preadipocytes were maintained in Dulbecco's modified Eagle's medium (DMEM) supplemented with 10% fetal bovine serum. Two days after confluence, 3T3-L1 cells were induced to differentiation using the standard adipogenic cocktail (0.5 mM IBMX, 1 μM dexamethasone and 10 μg/ml insulin) that was applied for 4 days and followed by 10 μg/ml insulin treatment for additional 4 days. The culture medium was changed every other day. The differentiated cells were maintained in normal serum containing DMEM. Mouse primary adipocytes were isolated from 10 to 12-week-old male C57BL/6J mice using a collagenase digestion protocol as described, briefly, white fat pads were removed, minced and digested using collagenase for 60-90 min at 37° C. in Krebs-Ringers phosphate HEPES buffer (KRPH) (15 mM HEPES, pH 7.4, 118 mM NaCl, 4.8 mM KCl, 1.2 mM MgSO₄, 1.3 mM CaCl₂, 1.2 mM KHPO₃, 0.1% BSA). The stromal vascular fraction was separated from the adipocytes and cultured using DMEM and differentiated the same as 3T3-L1 adipocytes as described above. For glucose uptake, differentiated 3T3-L1 adipocytes and primary adipocytes are plated at 10,000 cells/well in 96-well plates and fasted overnight with low glucose serum-free medium (DMEM with 0.25% BSA) DMEM and 2-NBDG (Cayman Chemical) was performed as follow: briefly, 3T3-L1 cells were washed twice with warm (37° C.) KRPH buffer, and adipocytes were either untreated or treated with different concentration of CYSTM1 (1 nM, 10 nM, 100 nM and 1000 nM) for 1 h in KRPH buffer containing 400 nM of 2-NBDG. NBDG uptake was measured using a fluorescent microplate reader (Thermo Scientific Varioskan LUX Multimode Microplate Reader). The efficacy of CYSTM1 and its potential inhibitors to stimulate glucose uptake in differentiated 3T3-L1 adipocytes was evaluated (FIGS. 8A and 8B) and mouse primary adipocytes (FIGS. 8C and 8D). A concentration-dependent increase was observed upon treatment with CYSTM1 in both adipocytes. The effect of CYSTM1 was blocked by co-treatment of ERK inhibitor (ERK1/2 inhibitor 1, MedChemExpress, 1 μM), AKT inhibitor (GSK690693, MedChemExpress, 1 μM), and AMPK inhibitor (Dorsomorphin, MedChemExpress, 1 μM) in both 3T3-L1 cells and primary adipocytes. These results suggest that the increase in glucose uptake by CYSTM1 is through partial activation of the phosphorylation of ERK, AKT and AMPK pathways.

In summary, insulin secretion from the B-cell of the endocrine pancreas is enhanced by the CYSTM1 administered in various forms such as oral gavage, i.p. injection, and via viral transfection. This pancreatic secretion mechanism involves a diversity of components such as glucose, amino acids, catecholamines, and peptides. The glucose uptake by adipocytes is also controlled by a complex network of metabolic factors. This network also includes a diversity of components such as insulin, GLP-1 and fibroblast growth factor 21 (FGF21). The specific liberation of CYSTM1 peptides in the stomach and partially in the pancreas suggests that the CYSTM1 peptides may be components of the gastroinsular axis for insulin release. By virtue of the induced glucose uptake in the adipose tissues, it is also suggested that the CYSTM1 peptides may be components of the gastro-adipose tissues axis for glucose uptake.

Although the invention has been described in terms of certain embodiments, other embodiments apparent to those of ordinary skill in the art are also within the scope of this invention. Accordingly, the scope of the invention is intended to be defined only by the claims which follow.

### INDUSTRIAL APPLICABILITY

The present compound or composition is potent in stimulating insulin secretion by pancreatic cells/tissue while lowering blood glucose by inducing glucose uptake by adipocytes in an insulin-independent manner, which is useful in treating both type I and type II diabetes and other failure in glucose uptake in the absence of endocrine pancreas in a subject.

### REFERENCES

The following references are cited herein, which are incorporated herein by reference:
1. Thiago M. Venancio, L. Aravind. (2010) CYSTM, a novel cysteine-rich transmembrane module with a role in stress tolerance across eukaryotes. Bioinformatics, Volume 26, Issue 2, 15 Jan. 2010, Pages 149-152.
2. Geng, Z., Liu, J., Hu, J., Wang, Y., Tao, Y., Zheng, F., Wang, Y., Fu, S., Wang, W., Xic, C., Zhang, Y., and Gong, F. (2020). Crucial transcripts predict response to initial immunoglobulin treatment in acute Kawasaki disease. Sci Rep 10, 17860.
3. Mastrokolias, A., Ariyurek, Y., Gocman, J. J., Van Duijn, E., Roos, R. A., Van Der Mast, R. C., Van Ommen, G. B., Den Dunnen, J. T., T Hoen, P. A., and Van Roon-Mom, W. M. (2015). Huntington's disease biomarker progression profile identified by transcriptome sequencing in peripheral blood. Eur J Hum Genet 23, 1349-1356.
4. Ou, S. I., Xiu, J., Nagasaka, M., Xia, B., Zhang, S. S., Zhang, Q., Swensen, J. J., Spetzler, D., Korn, W. M., Zhu, V. W., and Liu, S. V. (2021). Identification of Novel CDH1-NRG2alpha and F11R-NRG2alpha Fusions in NSCLC Plus Additional Novel NRG2alpha Fusions in Other Solid Tumors by Whole Transcriptome Sequencing. JTO Clin Res Rep 2, 100132.
5. Jun Du. Jinguo Wang (2021). CYSTM1: A Novel Biomarker for Hepatocellular Carcinoma Prognosis. https://doi.org/10.21203/rs.3.rs-150866/v1.

1. An isolated glucoregulatory compound having both insulinotropic and blood glucose lowering activities for treating a disease in a subject, the isolated glucoregulatory compound comprising protein, peptide, or protein fragment, or any combination thereof.

2. The isolated glucoregulatory compound of claim 1, wherein the isolated glucoregulatory compound is a glucoregulatory peptide derived from cysteine rich transmembrane module containing 1 (CYSTM1).

3. The isolated glucoregulatory compound of claim 1, wherein the protein, peptide, or protein fragment has at least 95% homology to an amino acid sequence of CYSTM1.

4. A pharmaceutical composition comprising the isolated glucoregulatory compound according to claim 1.

5. The pharmaceutical composition of claim 4, wherein the disease comprises type 1 diabetes, type 2 diabetes mellitus, or any failure in glucose uptake independent of insulin secretion by pancreatic cells in the subject.

6. The pharmaceutical composition of claim 4, further comprising a pharmaceutically acceptable carrier.

7. The pharmaceutical composition of claim 4, wherein the isolated glucoregulatory compound is represented by the following formula:
HN—X—CO—R′,
or a pharmaceutically acceptable salt or zwitterion thereof, wherein R′ is selected from H, OR, and —NRR;
R for each instance is independently selected from H, a lower branched alkyl group and an unbranched alkyl group;
X is a glucoregulatory peptide selected from an amino acid sequence of SEQ ID NO: 1 and SEQ ID NO: 2;
HN denotes an amine group at an amino terminus of the glucoregulatory peptide; and CO denotes a carbonyl group at a carboxyl terminus of the glucoregulatory peptide.

8. The pharmaceutical composition of claim 7, wherein the glucoregulatory peptide is further protected by one or more moieties such that the protected glucoregulatory peptide possesses higher insulinotropic and blood glucose lowering activities than those of an unprotected form thereof.

9. A method of treating a disease in a subject in need thereof, the method comprising administering a pharmaceutical composition comprising a therapeutically effective amount of the isolated glucoregulatory compound according to claim 1 to the subject.

10. The method of claim 9, wherein the disease includes type 1 diabetes, type 2 diabetes mellitus, or any failure in glucose uptake independent of insulin secretion by pancreatic cells in the subject.

11. The method of claim 9, wherein the subject includes human and other non-human mammals.

12. The method of claim 9, wherein the pharmaceutical composition comprises an effective amount of the isolated glucoregulatory compound such that an insulinotropic response is triggered and glucose uptake is enhanced in said subject by administering the composition to said subject.

13. The method of claim 9, wherein the composition further comprises a pharmaceutically acceptable carrier.

14. The method of claim 9, wherein the isolated glucoregulatory compound is represented by the following formula:
HN—X—CO—R′,
or a pharmaceutically acceptable salt or zwitterion thereof, wherein R′ is selected from H, OR, and —NRR;
R for each instance is independently selected from H, a lower branched alkyl group, and an unbranched alkyl group;
X is a glucoregulatory peptide selected from an amino acid sequence of SEQ ID NO: 1 and SEQ ID NO: 2;
HN denotes an amine group at an amino terminus of the glucoregulatory peptide; and CO denotes a carbonyl group at a carboxyl terminus of the glucoregulatory peptide.

15. The method of claim 9, wherein the pharmaceutical composition is administered via, or the pharmaceutical composition is formulated into a form suitable for an administration route including oral, intravenous, intramuscular, intraperitoneal, or subcutaneous administrations.

16. A composition comprising a nucleic acid encoding a glucoregulatory peptide derived from cysteine rich transmembrane module containing 1 (CYSTM1) for treating a disease in a subject.

17. The composition of claim 16, further comprising an expression vector to be administered with the nucleic acid to the subject in order to express the glucoregulatory peptide in said subject.

18. The composition of claim 16, wherein the nucleic acid encoding the glucoregulatory peptide derived from the CYSTM1 is selected from a nucleotide sequence of SEQ ID NO: 3, SEQ ID NO: 4, SEQ ID NO: 5, and SEQ ID NO: 6.

19. A method of treating a disease in a subject in need thereof, the method comprising administering a therapeutically effective amount of the composition according to claim 16 to the subject.

20. The method of claim 19, wherein the disease comprises hyperglycemia, type 1 diabetes, type 2 mellitus, or any failure in glucose uptake independent of insulin secretion by pancreatic cells.


SEQUENCE LISTING (6 sequences)

SEQ ID NO: 1 - protein, 97 aa, Homo sapiens
MNQENPPPYPGPGPTAPYPPYPPQPMGPGPMGGPYPPPQGYPYQGYPQYGWQGGPQEPPKTTVYVVEDQRRDELGPSTCLTACWTALCCCCLWDMLT

SEQ ID NO: 2 - protein, 104 aa, Mus musculus
MNPENPPPYPGPGPTAPYPPYPQQPMGPMGPMGAPPPQGYPYPPPQGYPYQGYPQYGWQGGPQEPPKTTVYVVEDQRRDDLGPSTCLTACWTALCCCCLWDMLT

SEQ ID NO: 3 - DNA, 294 bp, Homo sapiens
  source 1..294: mol_type = genomic DNA
atgaaccaagagaaccctccaccatatccaggccctggtccaacggccccatacccaccttatccaccacaaccaatgggtccaggacctatggggggaccctacccacctcctcaagggtacccctaccaaggatacccacagtacggctggcagggtggacctcaggagcctcctaaaaccacagtgtatgtggtagaagaccaaagaagagatgagctaggaccatccacctgcctcacagcctgctggacggctctctgttgctgctgtctctgggacatgctcacctga

SEQ ID NO: 4 - DNA, 315 bp, Mus musculus
  source 1..315: mol_type = genomic DNA
atgaacccagagaaccctccaccgtatccgggccccgggccaacagccccatacccaccttatccacaacagccaatggggccaatggggcctatgggagccccacctcctcaggggtacccctacccaccacctcaggggtacccctatcaaggatacccacagtacggctggcagggtggacctcaggagcctcctaagaccacagtgtatgtggtggaagaccaaagaagagacgacctgggcccatccacctgcctcacagcctgctggactgctctgtgttgctgctgcctctgggacatgctcacctga

SEQ ID NO: 5 - RNA, 294 nt, Homo sapiens
  source 1..294: mol_type = mRNA
atgaaccaagagaaccctccaccatatccaggccctggtccaacggccccatacccaccttatccaccacaaccaatgggtccaggacctatggggggaccctacccacctcctcaagggtacccctaccaaggatacccacagtacggctggcagggtggacctcaggagcctcctaaaaccacagtgtatgtggtagaagaccaaagaagagatgagctaggaccatccacctgcctcacagcctgctggacggctctctgttgctgctgtctctgggacatgctcacctga

SEQ ID NO: 6 - RNA, 315 nt, Mus musculus
  source 1..315: mol_type = mRNA
atgaacccagagaaccctccaccgtatccgggccccgggccaacagccccatacccaccttatccacaacagccaatggggccaatggggcctatgggagccccacctcctcaggggtacccctacccaccacctcaggggtacccctatcaaggatacccacagtacggctggcagggtggacctcaggagcctcctaagaccacagtgtatgtggtggaagaccaaagaagagacgacctgggcccatccacctgcctcacagcctgctggactgctctgtgttgctgctgcctctgggacatgctcacctga